(19) United States
(12) Patent Application Publication
(10) Pub. No.: US 2025/0353936 A1
(43) Pub. Date: Nov. 20, 2025

(54) CHEMICALLY MODIFIED HEPARIN

(71) Applicant: IHP Therapeutics Inc., San Carlos, CA (US)

(72) Inventors: John Eric Paderi, San Francisco, CA (US); Gabriel N. Njikang, Orcutt, CA (US)

(21) Appl. No.: 18/872,252

(22) PCT Filed: Jun. 6, 2023
(86) PCT No.: PCT/US2023/024568

§ 371 Date: Dec. 5, 2024

Related U.S. Application Data
(60) Provisional application No. 63/349,395, filed on Jun. 6, 2022.

(51) Int. Cl. C08B 37/00; A61K 31/727; A61P 35/00

(52) U.S. Cl. CPC C08B 37/0075; A61K 31/727; A61P 35/00

(57) ABSTRACT

Provided is a bovine intestinal heparin chemically modified with a 1-(3-dimethylaminopropyl)-3-ethylurea (EDU)-amide having anti-factor IIA activity is about 20 to about 30 IU/mg, wherein the modified bovine intestinal heparin exhibits between 20% and 50% of the anti-factor IIa activity of the nonchemically modified bovine intestinal heparin, as well as pharmaceutical compositions, compositions comprising chemically modified bovine intestinal heparin, and methods for making and using the same

### CROSS REFERENCE TO RELATED APPLICATIONS

This application claims the benefit under 35 U.S.C. § 119(e) to U.S. Provisional Application No. 63/349,395, filed Jun. 6, 2022, which is hereby incorporated by reference in its entirety.

### BACKGROUND

Sickle cell disease (SCD) is a devastating disease that affects over 100,000 people in the U.S. and more than 6 million worldwide. It is associated with incapacitating pain and chronic, progressive ischemic damage to almost every organ in the body, plummeting the life expectancy by more than 20 years. The hallmark of SCD is vaso-occlusive crisis (VOC). VOCs are excruciatingly painful acute events and serve as an antecedent to severe complications such as acute chest syndrome (ACS), a type of acute lung injury and a major cause of death among SCD patients. The resulting impact on patients is profound and impacts every aspect of life. While several prophylactic drugs such as anti-P-selectin antibody (crizanlizumab), hydroxyurea, or L-glutamine have shown promise in at least partially reducing VOC, no disease specific acute VOC therapy has ever been approved, representing a major treatment gap.

### SUMMARY

The present disclosure, in one embodiment, provides a chemically modified bovine intestinal heparin, wherein at least a portion of free carboxylic acid moieties on a non-chemically modified bovine intestinal heparin have been converted to a N-acylurea amide such that the chemically modified bovine intestinal heparin exhibits between 20% and 50% of the anti-factor IIa activity of the non-chemically modified bovine intestinal heparin; and wherein the complement inhibition activity of the chemically modified bovine intestinal heparin is substantially the same as that of the non-chemically modified bovine intestinal heparin, and pharmaceutical compositions comprising the same. In certain embodiments, the P-selectin inhibition activity is not less than 25% of the potency of non-chemically modified bovine intestinal heparin. The chemically modified bovine intestinal heparin disclosed herein, and compositions comprising the same, have decreased anticoagulant activity and are optimized for selectin and complement inhibition, allowing for effective therapeutic effect when administered to a subject in need thereof, and with limited risk for adverse bleeding.

In certain embodiments, provided is a method for treating or lessening one or more symptoms of sickle cell disease in a subject in need thereof, comprising administering to the subject an effective amount of the pharmaceutical composition as disclosed herein. In certain embodiments, the subject is in the early phase of vaso-occlusive crisis, such as the prodromal phase. In certain embodiments, the subject is in established vaso-occlusive crisis (VOC).

In certain embodiments, provided is a method for preventing or reversing cellular adhesion in a subject in need thereof, comprising administering to the subject an effective amount of the pharmaceutical composition as disclosed herein.

In certain embodiments, provided is a method for preventing or reversing complement activation in a subject in need thereof, comprising administering to the subject an effective amount of the pharmaceutical composition as disclosed herein.

In certain embodiments, provided is a method for treating a solid tumor in a subject in need thereof, comprising administering to the subject an effective amount of the pharmaceutical composition as disclosed herein. In certain embodiments, the solid tumor expresses at least one of sLex or sLea (Sialyl Lewis^{x} or Sialyl Lewis^{a}). In certain embodiments, the solid tumor is a gastrointestinal, breast, prostate, ovarian, colorectal, liver, lung, cervical, head, neck, esophageal, brain, or pancreatic tumor.

Also provided is a method for treating a disease or disorder mediated at least in part by inhibition of cell binding to P-selectin and/or inhibition of a complement activation pathway in a subject in need thereof, comprising administering to the subject an effective amount of a pharmaceutical composition comprising a chemically modified bovine intestinal heparin as described herein, or a composition comprising the same, wherein the disease or disorder is, but is not limited to, a cancer, a hematologic cancer, melanoma, leukemia, multiple myeloma, chemotherapy-induced peripheral neuropathy (CIPN), beta thalassemia, atypical hemolytic uremic syndrome (aHUS), paroxysmal nocturnal hemoglobinuria (PNH), a neurological disease, amyotrophic lateral sclerosis (ALS), sickle cell disease (including, but not limited to, vaso-occlusive crisis), immune response in gene therapy with adeno-associated virus (AAV), acute respiratory distress syndrome (ARDS), a cardiovascular disorder, an ophthalmological disease or disorder, a nephrological disorder, thrombogenic microangiopathy (TMA), hereditary angioedema, thrombotic thrombocytopenic purpura (TTP), Shiga toxin positive HUS, post-infection HUS, thrombotic microangiopathy, membranoproliferative glomerulonephritis (MPGN), primary MPGN, C3 glomerulopathy (C3G), transplant rejection, delayed kidney graft rejection, antibody-mediated kidney graft rejection, kidney graft reperfusion injury, kidney transplant in CAPS patients, neuromyelitis optica, multiple sclerosis, Guillain-Barré syndrome, myasthenia gravis, lupus nephritis, IgA nephropathy, rheumatoid arthritis, Crohn disease, ulcerative colitis, hemolytic anemia, autoimmune hemolytic anemia, pemphigus and pemphigoid, anti-phospholipid syndrome, cold agglutinin disease, severe thrombocytopenia, macular degeneration, uveitis, ANCA-associated vasculitis, atherosclerosis, mood disorders, asthma, chronic obstructive pulmonary disease (COPD), anaphylaxis, sepsis, cerebral malaria, psoriatic arthropathy, dermatomyositis, osteoarthritis, dementia, glaucoma, diabetic angiopathy, myocardial infarction, stroke, post-bypass, polytrauma, neurotrauma, antiphospholipid syndrome, preeclampsia, or hemodialysis. In certain embodiments, the treating comprises reducing inflammation or reducing or inhibiting an inflammatory response as a result of the disease or disorder.

### BRIEF DESCRIPTION OF DRAWINGS

FIG. 1 shows the effect of test compound on neutrophil cell binding to immobilized P-selectin in an in vitro model.

FIG. 2 shows complement-mediated hemolysis data for unmodified porcine heparin, Compound A and Compound C.

FIG. 3 shows body weight increased in all groups throughout the 70 day study period.

FIG. 4 shows tumor growth was inhibited by combination treatment with sensitizing dose of gemcitabine and test compound.

FIG. 5 shows ex vivo imaging of metastasis at day 70.

### DETAILED DESCRIPTION

All numerical designations, e.g., pH, temperature, time, concentration, and molecular weight, including ranges, are approximations which are varied (+) or (−) by increments of 0.1 or 10%. It is to be understood, although not always explicitly stated that all numerical designations are preceded by the term “about”. It also is to be understood, although not always explicitly stated, that the reagents described herein are merely exemplary and that equivalents of such are known in the art.

A “non-chemically modified” bovine intestinal heparin refers to a native bovine intestinal heparin which has not been modified by chemical means. Exemplary heparin which has been modified by chemical means, include, but are not limited to, LMWH derived from native heparin, heparin sulfate, biotechnology-derived heparin, synthetic heparin, or other heparin analogues. Exemplary chemical modifications include, but are not limited to, one or more of partial or full N- or O-desulfation, (e.g., 2-O-sulfated heparin, 3-O-sulfated heparin, 2,3-O-desulfated heparin, etc.), oxidation (e.g., periodate-oxidized heparin), reduction (e.g., reduction of heparin carboxyl groups, borohydride-reduced heparin, etc.), N-acetylation (including N-,O-desulfation followed by N-resulfation), sulfation, and the like.

A “chemically modified” bovine intestinal heparin refers to a non-chemically modified bovine intestinal heparin or native bovine intestinal heparin which has been modified to include a covalent bond to a 1-(3-dimethylaminopropyl)-3-ethylurea.

As used herein, a “1-(3-dimethylaminopropyl)-3-ethylurea (EDU)-amide” is an amide formed by reaction of 1-ethyl-3-(3-dimethylaminopropyl) carbodiimide (EDC, EDAC or EDCI) with a carboxylic acid (such as on a heparin). The reaction of EDAC with a carboxyl group generally proceeds through the addition of the free carboxylate to one of the double bonds of the diimide system to give an O-acylurea product. In the presence of a nucleophile, the acyl-nucleophile product is formed, plus the urea of the carbodiimide. In the absence of added nucleophiles, the O-acylurea rearranges to the more stable N-acylurea isomers shown below through an intramolecular acyltransfer:

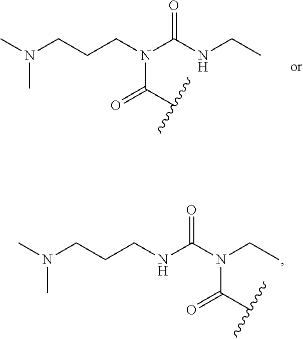

where the wavy line indicates a covalent bond to the heparin backbone.

In many cases, the chemically modified bovine intestinal heparin compounds of this disclosure are capable of forming acid and/or base salts by virtue of the presence of sulfoxides, and/or carboxyl groups, or groups similar thereto. In certain embodiments, provided are salts, compositions, dosage forms, or other materials which are useful in preparing a pharmaceutical composition that is suitable for veterinary or human pharmaceutical use.

A “pharmaceutical composition” is intended to include the combination of an active agent with a carrier, inert or active, making the composition suitable for diagnostic or therapeutic use in vitro, in vivo, or ex vivo.

A “subject” of diagnosis or treatment is an animal such as a mammal, including a human.

“An effective amount” refers to the amount of an agent sufficient to induce a desired biological and/or therapeutic result. That result can be alleviation of the signs, symptoms, or causes of a disease, or any other desired alteration of a biological system.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

“Administration” can be effected in one dose, continuously or intermittently throughout the course of treatment. Methods of determining the most effective means and dosage of administration are known to those of skill in the art and will vary with the pharmaceutical composition used for therapy, the purpose of the therapy, the target cell being treated, and the subject being treated. Single or multiple administrations can be carried out with the dose level and pattern being selected by the treating physician. Suitable dosage formulations and methods of administering the agents arc known in the art.

### Chemically-Modified Bovine Intestinal Heparin

Provided herein are chemically modified bovine intestinal heparins and compositions comprising the same. Heparin is a naturally occurring glycosaminoglycan. Glycosaminoglycans (GAGs) or mucopolysaccharides are long linear polysaccharides consisting of repeating disaccharide units. Except for keratan, the repeating unit consists of an amino sugar, along with a uronic sugar or galactose. Native heparins have a molecular weight ranging from 3 to 30 kDa. Various molecular weights for the heparin can be used herein, such as from a single disaccharide unit of about 650-700 Da, to a glycan of about 50 kDa. In some embodiments, the heparin is from about 10 to about 20 kDa. In some embodiments, the heparin is from about 15 to about 20 kDa. In some embodiments, the heparin is up to about 15, or about 16, or about 17, or about 18, or about 19, or about 20 kDa.

The main disaccharide units that occur in heparin include GlcA-GlcNAc, GlcA-GlcNS, IdoA-GlcNS, IdoA(2S)-GlcNS, IdoA-GlcNS(6S), and IdoA(2S)-GlcNS(6S). GlcA denotes β-D-glucuronic acid; IdoA denotes α-L-iduronic acid; IdoA(2S) denotes 2-O-sulfo-α-L-iduronic acid; GlcNAc denotes 2-deoxy-2-acetamido-α-D-glucopyranosyl; GlcNS denotes 2-deoxy-2-sulfamido-α-D-glucopyranosyl; and GlcNS(6S) denotes 2-deoxy-2-sulfamido-α-D-glucopyranosyl-6-O-sulfate. The most common disaccharide unit in heparin is composed of a 2-O-sulfated iduronic acid and 6-O-sulfated, N-sulfated glucosamine, IdoA(2S)-GlcNS(6S).

Heparin compounds and compositions having decreased anticoagulant activity can allow a higher dose of the heparin to be administered to a subject where anticoagulation activity is contraindicated (e.g., subjects taking aspirin, ibuprofen, or other anti-inflammatory medicines (e.g, NSAIDs) or medicines containing these ingredients). The anticoagulant activity of heparin can also be measured with respect to its activity to inhibit factor Xa (fXa) or factor IIa (thrombin). An example can be found in, e.g., Stuart, M, Johnson, L, Hanigan, S, Pipe, S W, Li, S-H. Anti-factor IIa (FIIa) heparin assay for patients on direct factor Xa (FXa) inhibitors. J Thromb Haemost. 2020; 00:1-8 (doi.org/10.1111/jth.14806) and the examples disclosed herein. The bovine intestinal heparin as used herein is derived from bovine intestine.

In certain embodiments, provided is a chemically modified bovine intestinal heparin, wherein at least a portion of free carboxylic acid moieties on a non-chemically modified bovine intestinal heparin have been converted to a N-acylurea amide such that the chemically modified bovine intestinal heparin exhibits between 20% and 50%, or from about 20% to about 40%, or from about 25% to 40%, or from about 25% to 35%, or about 20%, or about 25%, or about 30%, or about 35%, or about 40%, or about 45%, or about 50%, of the anti-factor IIa activity of the non-chemically modified bovine intestinal heparin; and wherein the complement inhibition activity of the chemically modified bovine intestinal heparin is substantially the same as that of the non-chemically modified bovine intestinal heparin.

In certain embodiments, provided is a pharmaceutical composition comprising a chemically modified bovine intestinal heparin, wherein at least a portion of free carboxylic acid moieties on a non-chemically modified bovine intestinal heparin have been converted to a N-acylurea amide such that the chemically modified bovine intestinal heparin exhibits between 20% and 50%, or from about 20% to about 40%, or from about 25% to 40%, or from about 25% to 35%, or about 20%, or about 25%, or about 30%, or about 35%, or about 40%, or about 45%, or about 50%, of the anti-factor IIa activity of the non-chemically modified bovine intestinal heparin; and wherein the complement inhibition activity of the chemically modified bovine intestinal heparin is substantially the same as that of the non-chemically modified bovine intestinal heparin, and a pharmaceutically acceptable excipient.

In certain embodiments, when compared herein, the “chemically modified” heparin and the “non-chemically modified” heparin are derived from the same source. In certain embodiments, when compared herein, the “chemically modified” heparin and the “non-chemically modified” heparin are not derived from the same source.

In certain embodiments, the non-chemically modified bovine intestinal heparin has an anti-factor IIa activity greater than 80 U/mg. In certain embodiments, the non-chemically modified bovine intestinal heparin has an anti-factor IIa activity greater than 90 U/mg. In certain embodiments, the non-chemically modified bovine intestinal heparin has an anti-factor IIa activity of about 100 U/mg, or from about 90 U/mg to about 135 U/mg.

In certain embodiments, the chemically modified bovine intestinal heparin has an anti-factor Ila activity less than 135 U/mg.

In certain embodiments, the chemically modified bovine intestinal heparin has an anti-factor Ila activity between 80 U/mg and 135 U/mg. In certain embodiments, the chemically modified bovine intestinal heparin has an anti-factor IIa activity between 90 U/mg and 135 U/mg.

In certain embodiments, the chemically modified bovine intestinal heparin exhibits about between 20% and 50%, or from about 20% to about 40%, or from about 25% to 40%, or from about 25% to 35%, or from about 25% to 30%, or from about 30% to 35%, or about 20%, or about 25%, or about 30%, or about 35%, or about 40%, or about 45%, or about 50%, of the anti-factor IIa activity of the non-chemically modified bovine intestinal heparin.

In certain embodiments, the chemically modified bovine intestinal heparin comprises one or more chemically modified saccharide units of Formula I:

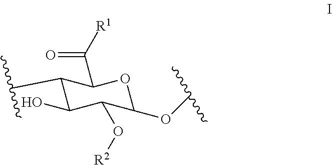

wherein:
each R¹ is independently

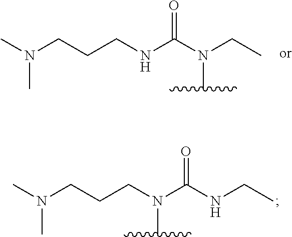

and
R² is hydrogen, —S(O)₂O⁻, —S(O)₂OH, or —S(O)₂OM; where M is a cation.

In certain embodiments, R² is hydrogen, —S(O)₂O⁻, —S(O)₂OH, or —S(O)₂ONa.

In certain embodiments, the chemically modified bovine intestinal heparin comprises one or more chemically modified saccharide units of Formula IIA:

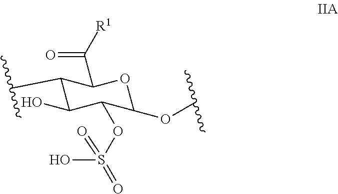

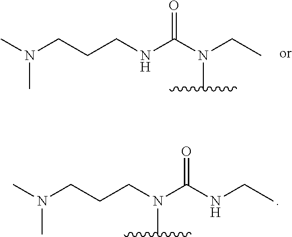

wherein each R¹ is independently

In certain embodiments, the chemically modified bovine intestinal heparin comprises one or more chemically modified saccharide units of Formula IIB:

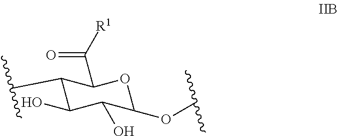

wherein each R¹ is independently

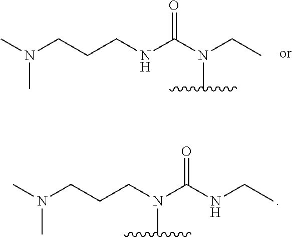

In certain embodiments, R¹ is

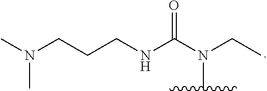 [SMILES: *N(CC)C(=O)NCCCN(C)C]

It is contemplated that the bovine intestinal chemically modified heparin disclosed herein comprises a mixture of the EDU-amine isomers described above.

In some embodiments, the non-chemically modified bovine intestinal heparin has an anti-factor IIa activity of greater than about 135 U/mg, greater than about 130 U/mg, greater than about 125 U/mg, greater than about 120 U/mg, greater than about 115 U/mg, greater than about 110 U/mg, greater than about 105 U/mg, greater than about 100 U/mg, greater than or about 90 U/mg, greater than about 85 U/mg, greater than about 80 U/mg, about 135 U/mg, about 130 U/mg, about 125 U/mg, about 120 U/mg, or about 115 U/mg, or about 110 U/mg, or about 105 U/mg, or about 100 U/mg, or about 90 U/mg, or about 85 U/mg, or about 80 U/mg, or about 80 U/mg to 135 U/mg, or about 90 U/mg to 135 U/mg, or about 90 U/mg to 120 U/mg, or about 90 U/mg to 110 U/mg, or about 80 U/mg to 120 U/mg, or about 80 U/mg to 110 U/mg, or about 100 U/mg to 135 U/mg, or about 100 U/mg to 130 U/mg, or about 100 U/mg to 125 U/mg, or about 100 U/mg to 120 U/mg, or about 100 U/mg to 130 U/mg, or about 100 U/mg to 120 U/mg, or about 100 U/mg to 110 U/mg, or about 110 U/mg to 140 U/mg, or about 110 U/mg to 130 U/mg, or about 110 U/mg to 120 U/mg, or about 120 U/mg to 140 U/mg, or about 120 U/mg to 130 U/mg, or about 130 U/mg to 140 U/mg.

In certain embodiments, the chemically modified heparin exhibits an anti-factor IIa activity of about 40 U/mg, or about 35 U/mg, or about 30 U/mg, or about 29 U/mg, or about 28 U/mg, or about 27 U/mg, or about 26 U/mg, or about 25 U/mg, or about 20 U/mg.

In certain embodiments, the chemically modified heparin exhibits an anti-factor IIa activity of about 25-35 U/mg. In certain embodiments, the chemically modified heparin exhibits an anti-factor Ila activity of about 25-30 U/mg.

In certain embodiments, provided herein is a chemically modified bovine intestinal heparin, wherein at least a portion of free carboxylic acid moieties on a non-chemically modified heparin having an anti-factor IIa activity greater than 90 U/mg, have been converted to an 1-(3-dimethylaminopropyl)-3-ethylurea (EDU)-amide such that the chemically modified bovine intestinal heparin exhibits about 20-30%, or about 25-30%, of the anti-factor IIa activity of the non-chemically modified bovine intestinal heparin.

In certain embodiments, provided herein is a pharmaceutical composition comprising chemically modified bovine intestinal heparin, wherein at least a portion of free carboxylic acid moieties on a non-chemically modified heparin having an anti-factor IIa activity greater than 90 U/mg, have been converted to an 1-(3-dimethylaminopropyl)-3-ethylurea (EDU)-amide such that the pharmaceutical composition exhibits about 20-30%, or about 25-30%, of the anti-factor IIa activity of the non-chemically modified bovine intestinal heparin, and a pharmaceutically acceptable excipient.

Also provided herein is a pharmaceutical composition comprising chemically modified heparin, wherein at least a portion of free carboxylic acid moieties on a non-chemically modified heparin having an anti-factor IIa activity greater than 90 U/mg, have been converted to an 1-(3-dimethylaminopropyl)-3-ethylurea (EDU)-amide such that the pharmaceutical composition exhibits about 20%, or about 25%, or about 27%, or about 28%, or about 29%, or about 30%, of the anti-factor Ila activity of the non-chemically modified heparin, and a pharmaceutically acceptable excipient.

In certain embodiments, the pharmaceutical composition increases the P-selectin inhibitory activity as compared to the non-chemically modified bovine intestinal heparin. In certain embodiments, the P-selectin inhibitory activity of the chemically modified bovine intestinal heparin is about 10% greater than non-chemically modified bovine intestinal heparin. In certain embodiments, the P-selectin inhibitory activity of the chemically modified bovine intestinal heparin is about 15%, or about 20%, or about 30%, or about 40%, or about 50%, or about 70%, or greater than about 100%, or greater than about 150%, or greater than about 200%, or greater than about 250%, or greater than about 300%, or greater than about 400%, of the non-chemically modified bovine intestinal heparin.

In certain embodiments, the P-selectin inhibitory activity (IC₅₀) is between less than 150% and greater than 150% that of the non-chemically modified bovine intestinal heparin. In certain embodiments, the P-selectin inhibitory activity (IC₅₀) is between less than 50% and greater than 50% that of the non-chemically modified bovine intestinal heparin.

In certain embodiments, the P-selectin inhibitory activity is substantially the same as that of the non-chemically modified bovine intestinal heparin (±50%, or ±40%, or ±30%, or ±20%, or ±10%).

In certain embodiments, the pharmaceutical composition does not diminish the complement inhibitory activity as compared to the non-chemically modified bovine intestinal heparin when measured by the CH50 assay as described herein. In certain embodiments, the complement inhibitory activity of the chemically modified heparin is about 100% of the non-chemically modified bovine intestinal heparin. In certain embodiments, the complement inhibitory activity of the chemically modified bovine intestinal heparin is about 95%, or about 90%, or about 85%, or about 80%, of the non-chemically modified bovine intestinal heparin.

In certain embodiments, the complement inhibitory activity (IC₅₀) is substantially the same as that of non-chemically modified bovine intestinal heparin. In certain embodiments, the complement inhibitory activity (IC₅₀) is up to 150% higher than that of the non-chemically modified bovine intestinal heparin.

In certain embodiments the complement inhibitory activity (IC₅₀) of the chemically modified heparin is less than 1.5-fold less potent, up to 1.5-fold less potent, or less than 2 times less potent, or about 2 times less potent, than that of the non-chemically modified bovine intestinal heparin.

In certain embodiments, the pharmaceutical composition, as compared to non-chemically modified bovine intestinal heparin, diminishes anticoagulant activity by up to 80%, and does not increase or decrease P-selectin inhibition activity by more than 150%.

In certain embodiments, the pharmaceutical composition, as compared to non-chemically modified bovine intestinal heparin, and diminishes anticoagulant activity by up to 80%, increases P-selectin inhibition activity by up to 150%.

In certain embodiments, the pharmaceutical composition, as compared to non-chemically modified bovine intestinal heparin, and diminishes anticoagulant activity by up to 80%, decreases P-selectin inhibition activity by up to 150%.

In certain embodiments, the pharmaceutical composition, as compared to non-chemically modified bovine intestinal heparin, and diminishes anticoagulant activity by up to 80%, and decreases complement inhibitory activity by up to 50%.

In certain embodiments, the pharmaceutical composition, as compared to non-chemically modified bovine intestinal heparin, does not increase or decrease P-selectin inhibition activity by more than 150%, and does not decrease complement inhibitory activity by more than 50%.

In certain embodiments, the pharmaceutical composition, as compared to non-chemically modified bovine intestinal heparin, increases P-selectin inhibition activity by up to 150%, and does not decrease complement inhibitory activity by more than 50%.

In certain embodiments, the pharmaceutical composition, as compared to non-chemically modified bovine intestinal heparin, decreases the P-selectin inhibition activity by up to 150%, and does not decrease complement inhibitory activity by more than 50%.

In certain embodiments, the pharmaceutical composition, as compared to non-chemically modified bovine intestinal heparin, diminishes anticoagulant activity by up to 80%, does not increase or decrease P-selectin inhibition activity by more than 150%, and does not decrease complement inhibitory activity by more than 50%.

In certain embodiments, the pharmaceutical composition, as compared to non-chemically modified bovine intestinal heparin, diminishes anticoagulant activity by up to 80%, increases P-selectin inhibition activity by up to 150%, and does not decrease complement inhibitory activity by more than 50% when measured by the CH50 assay as described herein.

In certain embodiments, the pharmaceutical composition, as compared to non-chemically modified bovine intestinal heparin, diminishes anticoagulant activity by up to 80%, decreases the P-selectin inhibition activity by up to 150%, and does not decrease complement inhibitory activity by more than 50%.

In certain embodiments, the chemically modified bovine intestinal heparin is unfractionated bovine intestinal heparin.

In certain embodiments, the chemically modified bovine intestinal heparin disclosed herein is comprised of heparin which has been reacted with 1-ethyl-3-(3-dimethylaminopropyl) carbodiimide (EDC, EDAC or EDCI) in the absence of a nucleophile, and thus the EDAC is not being used to conjugate the heparin to another compound or material. Further, the degree, or level, of sulfation on the bovine heparin is not reduced or modified by the chemical modification (i.e., EDU modification). In addition, the molecular weight of the bovine heparin is not reduced or modified by the chemical modification (i.e., EDU modification).

In certain embodiments, provided is a chemically modified bovine intestinal heparin comprising from about 15 to about 90 disaccharide units, wherein up to about 15%, or about 2.5% to 15%, or about 2.5% to 14% of the disaccharide units comprise a 1-(3-dimethylaminopropyl)-3-ethylurea (EDU)-amide.

In certain embodiments, provided is a chemically modified bovine intestinal heparin comprising from about 15 to about 90 disaccharide units, wherein up to about 15%, or about 2.5% to 15% of the disaccharide units comprise a 1-(3-dimethylaminopropyl)-3-ethylurea (EDU)-amide; and the anti-factor IIA activity from about 20 to about 30 IU/mg.

In certain embodiments, the anti-factor IIA activity is greater than 20 IU/mg, and less that the non-chemically modified bovine intestinal heparin.

In certain embodiments, the anti-factor IIA activity is between 17 and 50 IU/mg, or between 20 and 50 IU/mg, or between 20 and 45 IU/mg, or between 20 and 40 IU/mg, or between 20 and 35 IU/mg, or between 20 and 30 IU/mg, or about 25 IU/mg, 26 IU/mg, 27 IU/mg, or 28 IU/mg.

In certain embodiments, the P-selectin activity of the chemically modified bovine intestinal heparin is not substantially different than the parent non-chemically modified bovine intestinal heparin.

In certain embodiments, the P-selectin IC₅₀ of the chemically modified bovine intestinal heparin is less than about 3 μg/mL.

In certain embodiments, provided is a chemically modified bovine intestinal heparin of Formula IA:

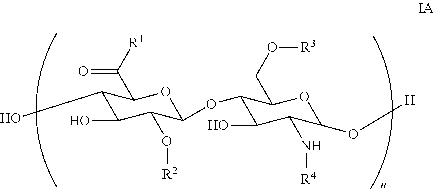

or a salt thereof, wherein:
n is 26-30;
each R¹ is independently —OH,

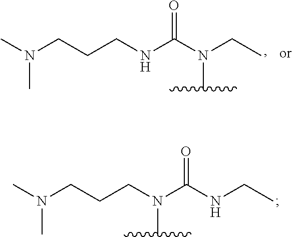

each R² is independently hydrogen, —S(O)₂O⁻, —S(O)₂OH, or —S(O)₂OM;
each R³ is independently hydrogen, —S(O)₂O⁻, —S(O)₂OH, or —S(O)₂OM;
each R⁴ is independently hydrogen, —S(O)₂O⁻, —S(O)₂OH, or —S(O)₂OM; and
each M is independently a cation;
wherein less than 15% of the R¹ moieties are either

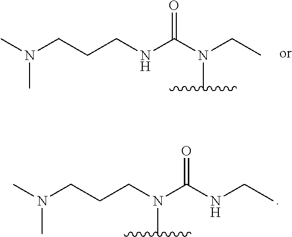

In certain embodiments, provided is a composition comprising a chemically modified bovine intestinal heparin as described herein.

In certain embodiments, provided is a chemically modified bovine intestinal heparin comprised of a bovine intestinal heparin, wherein less than 15%, or from 1% to 15%, or from 2.5% to 15%, or from 2.5% to 14% of carboxylic acid functional groups on the bovine intestinal heparin have a 1-(3-dimethylaminopropyl)-3-ethylurea (EDU)-amide moiety covalently bonded thereto. In certain embodiments, from about 2.5% to 15%, or from 5% to 15%, or from 5% to 14%, or from 8% to 14%, or from 9% to 14%, or from 10% to 15%, or about 13%, of carboxylic acid functional groups on the bovine intestinal heparin have a 1-(3-dimethylaminopropyl)-3-ethylurea (EDU)-amide moiety covalently bonded thereto.

In any of the embodiments described herein, the number of 1-(3-dimethylaminopropyl)-3-ethylurea (EDU)-amide groups per heparin is an average, wherein certain chemically modified bovine intestinal heparin compounds in a composition may have more 1-(3-dimethylaminopropyl)-3-ethylurea (EDU)-amide groups per heparin and others have less. Accordingly, in certain embodiments, the number of 1-(3-dimethylaminopropyl)-3-ethylurea (EDU)-amide groups as described herein is an average in a composition of chemically modified bovine intestinal heparin.

For example, in certain embodiments, the chemically modified bovine intestinal heparin is a composition where the average number of 1-(3-dimethylaminopropyl)-3-ethylurea (EDU)-amide groups per heparin, wherein the heparin has an average molecular weight of about 16 kDa, is about 3-5, or about 3-4, or about 3, or about 3.3, or about 3.6, or about 3.9, or about 4, or about 4.3, or about 4.5, or about 5, or 3.3-3.9. In certain embodiments, the average number of disaccharide units in the bovine heparin is 26-30, or about 26, or about 27, or about 28, or about 29, or about 30. In certain embodiments, the average number of disaccharide units in the bovine heparin is 26-30, and the average number of 1-(3-dimethylaminopropyl)-3-ethylurea (EDU)-amide groups per heparin is about 3-5, or about 3-4, or about 3, or about 3.3, or about 3.6, or about 3.9, or about 4, or about 4.3, or about 4.5, or about 5, or 3.3-3.9.

In certain embodiments, the chemically modified bovine intestinal heparin can be defined by the number of carboxylic acid functional groups which have been converted to a 1-(3-dimethylaminopropyl)-3-ethylurea (EDU)-amide, i.e., the degree of substitution (DOS) or percent functionalization. The degree of substitution (DOS) or percent functionalization is based on the percent of disaccharide units in a heparin which are functionalized with a 1-(3-dimethylaminopropyl)-3-ethylurea (EDU)-amide on the heparin backbone.

The total number of available disaccharide units present on the heparin can be calculated by dividing the molecular weight (or the average molecular weight) of a single disaccharide unit (e.g., about 500-600 Da, or about 575 Da) by the molecular weight of the glycan (e.g., about 15-17 kDa, or about 16 kDa). In certain embodiments, provided is a chemically modified bovine intestinal heparin, wherein the degree of substitution (DOS) or percent functionalization is less than 15%, or from about 1% to 15%, or from about 1.5% to 15%, or from about 2% to 15%, or from about 2.5% to 15%, or from about 3% to 14%, or from about 4% to 14%, or from about 5% to 14%, or from about 5% to about 13.5%, or from about 6% to about 13.5%, or from about 7% to about 13.5%, or from about 8% to about 13.5%, or from about 9% to about 13.5%, or from about 10% to about 13.5%, or about 11%, or about 13.5%, or about 13%.

In certain embodiments, the number of carboxylic acid functional groups which have been converted to a 1-(3-dimethylaminopropyl)-3-ethylurea (EDU)-amide may be described as a “percent functionalization by mass” based on the number of 1-(3-dimethylaminopropyl)-3-ethylurea (EDU)-amide units on the heparin backbone. In certain embodiments, provided is a chemically modified bovine intestinal heparin, wherein the percent functionalization by mass is less than 5%, about 0.1-4.5%, or about 1-4.5%, or about 2-4%, or about 3-4%, or about 3.5-4%, or about 3%, or about 3.5%, or about 3.6%, or about 3.7%, or about 3.8%, or about 4%.

In certain embodiments, provided is a chemically modified heparin of Formula IA:

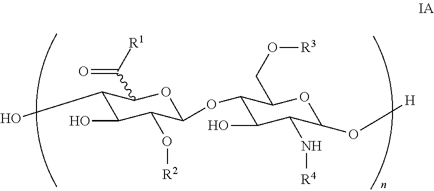

or a salt thereof, wherein:
n is 15-90;
R¹ is as defined herein;
each R² is independently hydrogen, —S(O)₂O⁻, —S(O)₂OH, or —S(O)₂OM;
each R³ is independently hydrogen, —S(O)₂O⁻, —S(O)₂OH, or —S(O)₂OM;
each R⁴ is independently hydrogen, —S(O)₂O⁻, —S(O)₂OH, or —S(O)₂OM; and
each M is independently a cation.

In certain embodiments, provided is a chemically modified heparin comprising from about 15 to about 90 disaccharide units, wherein about 1% to less than 15% of the disaccharide units comprise a 1-(3-dimethylaminopropyl)-3-ethylurea (EDU)-amide; and the anti-factor IIA activity is from about 20 IU/mg to about 30 IU/mg.

In certain embodiments, the heparin is from 9-50 KDa, or from about 9-35 KDa, or about 9 KDa, or about 12 KDa, or about 16 KDa, or about 20 KDa, or about 35 KDa, or about 50 KDa. In certain embodiments, n is about 15-87, or about 20-65, or about 25-35, or about 25-30.

In certain embodiments, provided is a chemically modified bovine intestinal heparin of Formula IA:

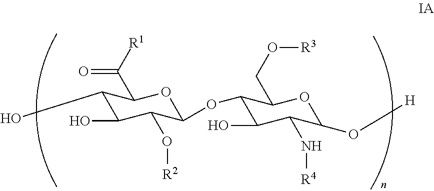

or a salt thereof, wherein:
n is 26-30;
R¹ is as defined herein;
R² is hydrogen, —S(O)₂O⁻, —S(O)₂OH, or —S(O)₂OM;
each R³ is independently hydrogen, —S(O)₂O⁻, —S(O)₂OH, or —S(O)₂OM;
each R⁴ is independently hydrogen, —S(O)₂O⁻, —S(O)₂OH, or —S(O)₂OM; and
each M is independently a cation.

In certain embodiments, provided is a chemically modified bovine intestinal heparin of Formula IA:

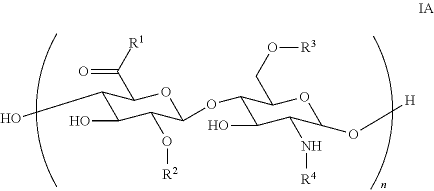

or a salt thereof, wherein:
n is 26-30;
each R¹ is independently —OH,

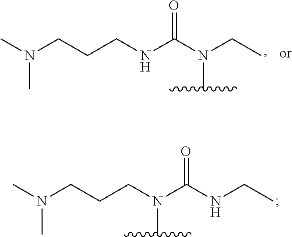

R² is hydrogen, —S(O)₂O⁻, —S(O)₂OH, or —S(O)₂OM;
each R³ is independently hydrogen, —S(O)₂O⁻, —S(O)₂OH, or —S(O)₂OM;
each R⁴ is independently hydrogen, —S(O)₂O⁻, —S(O)₂OH, or —S(O)₂OM; and
each M is independently a cation; wherein:
i) less than 15%, or from about 1% to 15%, or from about 1.5% to 15%, or from about 2% to 15%, or from about 2.5% to 15%, or from about 3% to 14%, or from about 4% to 14%, or from about 5% to 14%, or from about 5% to about 13.5%, or from about 6% to about 13.5%, or from about 7% to about 13.5%, or from about 8% to about 13.5%, or from about 9% to about 13.5%, or from about 10% to about 13.5%, or about 11%, or about 13.5%, or about 13%, of the R¹ moieties are selected from

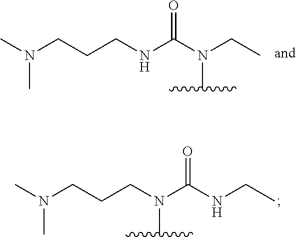

ii) less than 5, or from about 1 to 5, or from about 2 to 5, or from about 2.5 to 4.5, or about 2.5, or about 3, or about 3.5, or about 4, or about 4.5, of the R¹ moieties together are selected from

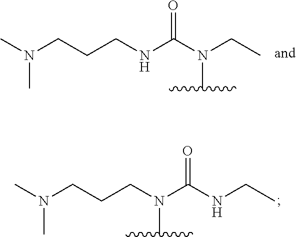

iii) about ⅞×n of the R¹ moieties are —OH.

In certain embodiments of Formula IA, each R¹ is independently —OH,

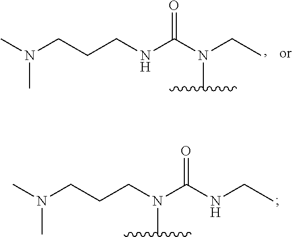

provided that less than 15%, or from about 1% to 15%, or from about 1.5% to 15%, or from about 2% to 15%, or from about 2.5% to 15%, or from about 3% to 14%, or from about 4% to 14%, or from about 5% to 14%, or from about 5% to about 13.5%, or from about 6% to about 13.5%, or from about 7% to about 13.5%, or from about 8% to about 13.5%, or from about 9% to about 13.5%, or from about 10% to about 13.5%, or about 11%, or about 13.5%, or about 13%, of the R¹ moieties are selected from

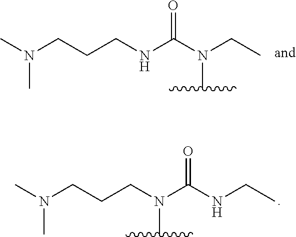

In certain embodiments of Formula IA, each R¹ is independently —OH,

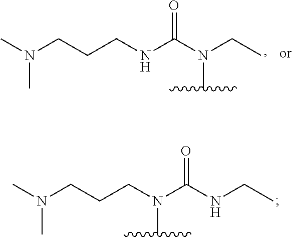

provided that less than 5, or from about 1 to 5, or from about 2 to 5, or from about 2.5 to 4.5, or about 2.5, or about 3, or about 3.5, or about 4, or about 4.5, of the R¹ moieties are selected from

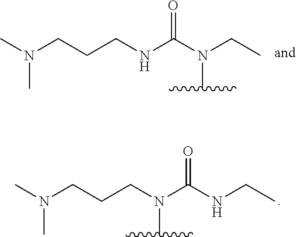

In certain embodiments of Formula IA, about ⅞×n of the R¹ moieties are —OH.

In certain embodiments, n is 26. In certain embodiments, n is 27. In certain embodiments, n is 28. In certain embodiments, n is 29. In certain embodiments, n is 30.

In certain embodiments, each R¹ is independently —OH or

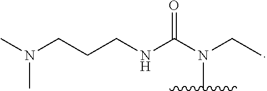 [SMILES: *N(CC)C(=O)NCCCN(C)C]

### Methods

Heparin, as well as various other chemically or biochemically modified heparins, have proved useful for many diseases and disorders. Chemically modified heparin as described herein, and compositions comprising the chemically modified heparin as described herein, which have decreased to no anti-thrombin activity, can be useful in many diseases and disorders for which heparin is administered, as well as the diseases and disorders for which heparin compositions having some or significant anti-factor IIa activity would be contraindicated.

In certain embodiments, provided is a method for reducing inflammation in a subject in need thereof, comprising administering to the subject an effective amount of a pharmaceutical composition comprising a chemically modified heparin as described herein.

TNF-alpha is a hallmark cytokine of inflammation and an important therapeutic target (see, e.g., Esposito, et al. Current medicinal chemistry 16.24 (2009): 3152-3167). It is contemplated that the chemically modified bovine intestinal heparin described herein would be capable of decreasing the production of TNF-alpha. Therefore, in certain embodiments, provided is a method for inhibiting the production of TNF-alpha in a subject in need thereof, comprising administering to the subject an effective amount of a pharmaceutical composition comprising a chemically modified heparin as described herein.

In certain embodiments, provided is a method for reducing anti-inflammatory properties in a subject, such as a reduction of leukocyte (such as neutrophil) recruitment to the endothelium by P- and L-selectin blockade, attenuation of cytokine production through NF-kB inhibition, inhibition of complement activation, or modulation of neutrophil extracellular traps (NETs) in a subject in need thereof, comprising administering to the subject an effective amount of a pharmaceutical composition comprising a chemically modified heparin as described herein.

In certain embodiments, provided is a method for treating or lessening one or more symptoms of acute respiratory distress syndrome (ARDS) in a subject in need thereof, comprising administering to the subject an effective amount of a pharmaceutical composition comprising a chemically modified heparin as described herein. In certain embodiments, the acute respiratory distress syndrome (ARDS) is a symptom of one or more of sepsis, SARS-CoV-2 infection, aspirating vomit, a near-drowning episode, severe pneumonia, physical damage the lungs, physical injury to the portion of the brain that controls breathing, pancreatitis (inflammation of the pancreas), massive blood transfusion, a burn, or inhalation of smoke or chemical fumes.

In certain embodiments, provided is a method for treating sepsis in a subject in need thereof, comprising administering to the subject an effective amount of a pharmaceutical composition comprising a chemically modified heparin as described herein.

In one embodiment, the subject is hospitalized. In one embodiment, the subject is treated with mechanical ventilation. In one embodiment, the subject suffers from a cytokine release syndrome.

P-selectin is an important therapeutic target for multiple diseases, including cancer, cardiovascular disorders and sickle cell disease (SCD) (see, e.g., Ataga, et al. New England Journal of Medicine 376.5 (2017): 429-439; Yeini, et al. Nature communications 12.1 (2021): 1-22; Borsig, Glycobiology 28.9 (2018): 648-655; Ludwig, et al. Expert opinion on therapeutic targets 11.8 (2007): 1103-1117; and Merle, et al. Proceedings of the National Academy of Sciences 116.13 (2019): 6280-6285). In certain embodiments, the compounds disclosed herein inhibit P-selectin mediated inflammatory response (such as to LPS) (see, e.g., Mayadas, et al. Cell 74.3 (1993): 541-554).

In certain embodiments, provided is a method for treating a cancer in a subject in need thereof, comprising administering to the subject an effective amount of a pharmaceutical composition comprising a chemically modified heparin as described herein. Exemplary cancer includes, but are not limited to, leukemia, multiple myeloma, gastrointestinal, breast, prostate, ovarian, colorectal, liver, lung, cervical, head, neck, melanoma, or pancreatic cancer.

In certain embodiments, provided is a method for treating a solid tumor in a subject in need thereof, comprising administering to the subject an effective amount of a pharmaceutical composition comprising a chemically modified heparin as described herein. Exemplary solid tumors include, but are not limited to, a glioblastoma, metastatic gastrointestinal, breast, prostate, ovarian, colorectal, liver, lung, cervical, head, neck, esophageal, brain, or pancreatic tumors.

In certain embodiments, the cancer is one with a high selectin ligand Sialyl Lewis, Sialyl Lewis X or Sialyl Lewis A (also known as CA19-9) expression. In certain embodiments, provided is a method for treating cancer in a subject in need thereof, where the subject exhibits expression or overexpression of Sialyl Lewis X or Sialyl Lewis A. Overexpression of Sialyl Lewis X or Sialyl Lewis A can be detected by measuring various biomarkers, such as antibodies.

In certain embodiments, the solid tumor is a CNS cancer, such as, but not limited to, a glioblastoma.

In certain embodiments, the tumor is colorectal, liver, pancreas, lung, breast, prostate, ovarian, head and neck, esophageal, or other tumor types.

In certain embodiments, provided is a method for inhibiting metastasis of cancer cells in a subject in need thereof, comprising administering to the subject an effective amount of a pharmaceutical composition comprising a chemically modified heparin as described herein. In certain embodiments, the cancer cells are melanoma cells. Melanoma, the most aggressive form of skin cancer, is often incurable once the cancer has metastasized. It has been shown that melanoma can metastasize via blood or lymphatic system. In certain embodiments, provided is a method for inhibiting metastasis of cancer cells to the mesentery. In certain embodiments, provided is a method for inhibiting metastasis of cancer cells to the lungs.

In certain embodiments, provided is a method for treating a cancer in a subject in need thereof, comprising administering to the subject an effective amount of a pharmaceutical composition comprising a chemically modified heparin as described herein. Exemplary cancers include, but are not limited to, hematologic cancers such as leukemia, multiple myeloma, and the like.

In certain embodiments, provided is a method for treating a disease or disorder mediated at least in part by inhibition of cell binding to P-selectin in a subject in need thereof, comprising administering to the subject an effective amount of a pharmaceutical composition comprising a chemically modified heparin as described herein. In certain embodiments, the treating comprises reducing inflammation or reducing or inhibiting an inflammatory response as a result of the disease or disorder. In certain embodiments, the treating comprises reducing P-selectin mediated sickle cell vaso-occlusive crisis.

In certain embodiments, the disease or disorder is a cancer (e.g., a hematologic cancer such as leukemia, multiple myeloma, and the like, or a metastatic cancer, such as melanoma, and the like), chemotherapy-induced peripheral neuropathy (CIPN), beta thalassemia, atypical hemolytic uremic syndrome (aHUS), paroxysmal nocturnal hemoglobinuria (PNH), a neurological disease such as amyotrophic lateral sclerosis (ALS), sickle cell disease (including, but not limited to vaso-occlusive crisis), immune response in gene therapy with adeno-associated virus (AAV), acute respiratory distress syndrome (ARDS), a cardiovascular disorder (e.g., post-myocardial infarction or interventional procedure), an ophthalmological disease or disorder, a nephrological disorder, or thrombogenic microangiopathy (TMA).

In certain embodiments, provided is a method for treating a vaso-occlusive crisis (VOC) in a subject in need thereof, comprising administering to the subject an effective amount of a pharmaceutical composition comprising a chemically modified heparin as described herein. In certain embodiments, the subject is in the early stages of VOC, such as the prodromal stage. In certain embodiments, the administration is via subcutaneous administration, such as at home or in a pharmacy, during the early phase of VOC. The early phase of VOC often precedes established VOC by one or more days, such as 1 day, 2 days, 3 days, 4 days, 5 days, from 1 to 5 days, from 1 to 4 days, from 2 to 5 days, from 2 to 4 days, from 1 to 3 days, or from 2 to 3 days. In certain embodiments, the administration is during a VOC, or in the early symptoms of a VOC, or in the established phase of VOC. Administration may alleviate or prevent one or more symptoms of VOC, such as but not limited to, a pain crisis, tissue injury, or hospitalization.

In certain embodiments, provided is a method for treating or lessening one or more symptoms of sickle cell disease in a subject in need thereof, comprising administering to the subject an effective amount of a pharmaceutical composition comprising a chemically modified heparin as described herein. In certain embodiments, the subject is in vaso-occlusive crisis. In certain embodiments, the subject in need thereof is in the prodromal, or early, phase of vaso-occlusive crisis (VOC).

In certain embodiments, the term administering includes self-administration, including self-administering outside of a healthcare setting, such as in a home setting.

In certain embodiments, provided is a method for treating a disease or disorder mediated at least in part by inhibition of a complement activation pathway in a subject in need thereof, comprising administering to the subject an effective amount of a pharmaceutical composition comprising a chemically modified heparin as described herein, or a composition comprising the same. Exemplary diseases or disorders can be found, for example, in Oberkersch, et al. Thrombosis research 125.5 (2010): e240-e245, and Morgan, et al. Nature reviews Drug discovery 14.12 (2015): 857-877.

Exemplary diseases or disorders include, but are not limited to, hereditary angioedema, paroxysmal nocturnal hemoglobinuria (PNH), chemotherapy-induced peripheral neuropathy (CIPN), beta thalassemia, atypical hemolytic uremic syndrome (aHUS), thrombotic thrombocytopenic purpura (TTP), Shiga toxin positive HUS, post-infection HUS, thrombotic microangiopathy, membranoproliferative glomerulonephritis (MPGN) such as primary MPGN, C3 glomerulopathy (C3G), transplant rejection, delayed kidney graft rejection, antibody-mediated kidney graft rejection, kidney graft reperfusion injury, kidney transplant in CAPS patients, neuromyelitis optica, multiple sclerosis, Guillain-Barré syndrome, myasthenia gravis, lupus nephritis, IgA nephropathy, rheumatoid arthritis, Crohn disease, ulcerative colitis, hemolytic anemia, autoimmune hemolytic anemia, pemphigus and pemphigoid, anti-phospholipid syndrome, cold agglutinin disease, severe thrombocytopenia, macular degeneration, uveitis, ANCA-associated vasculitis, atherosclerosis, mood disorders, asthma, chronic obstructive pulmonary disease (COPD), anaphylaxis, sepsis, acute respiratory distress syndrome (ARDS), cerebral malaria, psoriatic arthropathy, dermatomyositis, osteoarthritis, dementia, glaucoma, diabetic angiopathy, myocardial infarction, stroke, post-bypass, polytrauma, neurotrauma, antiphospholipid syndrome, preeclampsia, or hemodialysis. In certain embodiments, the treating comprises reducing inflammation or reducing or inhibiting an inflammatory response as a result of the disease or disorder.

AAV gene therapy has already demonstrated great promise in transforming disease management, yet several key barriers exist. Immune response to AAV administration results in 1) potential significant adverse events, and 2) production of AAV neutralizing antibodies, precluding re-administration of AAV therapy. While the immune response to AAV and associated pathologies are not yet fully elucidated, the innate immune system, especially complement activation, has emerged as the key driver. Therapeutically modulating complement during AAV administration thus has the potential to address both of these issues, improving the safety of AAV therapy while also greatly expanding its potential by allowing for multiple doses. With respect to safety, several adverse events have been observed clinically with AAV therapies, some of which have led to FDA clinical hold. Adverse events include atypical hemolytic uremic syndrome (aHUS) or other thrombogenic microangiopathies (TMA), which can have fatal consequence. Complement activation is emerging as a key driver of these adverse immune responses.

In certain embodiments, provided herein is a method for attenuating an immune response, such as an innate immune response, to AAV gene therapy, comprising administering to a subject in need thereof an effective amount of a chemically modified heparin as described herein, or a composition comprising the same. In certain embodiments, the method reduces neutralizing antibody production and/or a complement-mediated adverse response to AAV gene therapy.

In certain embodiments, provided herein is a chemically modified heparin for attenuating an immune response, such as an innate immune response, to AAV gene therapy. In certain embodiments, the chemically modified heparin reduces neutralizing antibody (NAb) production and/or a complement-mediated adverse response to AAV gene therapy.

In certain embodiments, provided herein is the use of a chemically modified heparin as disclosed herein in AAV gene therapy to attenuate the innate immune response, thereby 1) preventing or treating adverse events such as aHUS and TMA, and 2) reducing neutralizing antibody production and allowing for AAV re-administration.

In certain embodiments, provided herein is a method for treating a hemolytic disease as described herein, or one or more adverse effects from gene therapy, comprising administering to a patient in need thereof, an effective amount of a chemically modified heparin as disclosed herein, or a heparin having reduced anticoagulant activity relative to porcine unfractionated heparin, such as, but not limited to, glycol-split heparin (e.g., sevuparin, tafoxiparin, necuparanib, etc.), a 2-O, 3-O desulfated heparin (also referred to as ODSH or DSTAT), or an N-acetylated glycol-split heparin (e.g., roneparstat). In certain embodiments, the hemolytic disease is a cancer, a hematologic cancer, melanoma, leukemia, multiple myeloma, beta thalassemia, atypical hemolytic uremic syndrome (aHUS), paroxysmal nocturnal hemoglobinuria (PNH), a neurological disease, amyotrophic lateral sclerosis (ALS), sickle cell disease (including, but not limited to, vaso-occlusive crisis), immune response in gene therapy with adeno-associated virus (AAV), acute respiratory distress syndrome (ARDS), a cardiovascular disorder, an ophthalmological disease or disorder, a nephrological disorder, thrombogenic microangiopathy (TMA), hereditary angioedema, thrombotic thrombocytopeni purpura (TTP), Shiga toxin positive HUS, post-infection HUS, thrombotic microangiopathy, membranoproliferative glomerulonephritis (MPGN), primary MPGN, C3 glomerulopathy (C3G), transplant rejection, delayed kidney graft rejection, antibody-mediated kidney graft rejection, kidney graft reperfusion injury, kidney transplant in CAPS patients, neuromyelitis optica, multiple sclerosis, Guillain-Barré syndrome, myasthenia gravis, lupus nephritis, IgA nephropathy, rheumatoid arthritis, Crohn disease, ulcerative colitis, hemolytic anemia, autoimmune hemolytic anemia, pemphigus and pemphigoid, anti-phospholipid syndrome, cold agglutinin disease, severe thrombocytopenia, macular degeneration, uveitis, ANCA-associated vasculitis, atherosclerosis, mood disorders, asthma, chronic obstructive pulmonary disease (COPD), anaphylaxis, sepsis, cerebral malaria, psoriatic arthropathy, dermatomyositis, osteoarthritis, dementia, glaucoma, diabetic angiopathy, myocardial infarction, stroke, post-bypass, polytrauma, neurotrauma, antiphospholipid syndrome, preeclampsia, or hemodialysis. In certain embodiments, the hemolytic disease is selected from hemolytic uremic syndrome (HUS), atypical HUS (aHUS), paroxysmal nocturnal hemoglobinuria (PNH), sickle cell disease, thrombogenic microangiopathy, hemolytic anemia, autoimmune hemolytic anemia, and other conditions that cause hemolysis (such as hemodialysis).

### Administration and Dosing

The compounds and compositions disclosed herein can be used in the manufacture of medicaments and for the treatment of humans and other animals by administration in accordance with conventional procedures, such as an active ingredient in pharmaceutical compositions.

A compound of the present disclosure can be administered for therapy by any suitable route, specifically by oral or parental (including subcutaneous, intramuscular, intravenous, intravitreal, intrathecal, and intradermal) administration. It will also be appreciated that the preferred route will vary with the condition and age of the subject, and the disease being treated.

In one embodiment, the chemically modified heparin is administered in a composition. The present disclosure provides compositions comprising a chemically modified heparin and a pharmaceutically acceptable carrier. Pharmaceutically acceptable carriers known to one having ordinary skill in the art may be used, including water or saline. As is known in the art, the components as well as their relative amounts are determined by the intended use and method of delivery. Diluent or carriers employed in the compositions can be selected so that they do not diminish the desired effects of the chemically modified heparin. Examples of suitable compositions include aqueous solutions, for example, a solution in isotonic saline, 5% glucose. Other well-known pharmaceutically acceptable liquid carriers such as alcohols, glycols, esters and amides, may be employed. In certain embodiments, the composition further comprises one or more excipients, such as, but not limited to ionic strength modifying agents, osmolality modifying agents, solubility enhancing agents, sugars such as mannitol or sorbitol, pH buffering agent, surfactants, stabilizing polymer, preservatives, and/or co-solvents.

Suitable ionic strength modifying agents and/or osmolality modifying agents include, for example, glycerin, propylene glycol, mannitol, glucose, dextrose, sorbitol, sodium chloride, potassium chloride, and other electrolytes.

In certain embodiments, the composition is an aqueous solution. Aqueous solutions are suitable for use in composition formulations based on ease of formulation, as well as an ability to easily administer such compositions by means of instilling in the solution. In certain embodiments, the compositions are suspensions, viscous or semi-viscous gels, or other types of solid or semi-solid compositions. In certain embodiments, the composition is a solution that is directly applied to or contacts the internal wall of a vein or artery. In some embodiments, the composition comprises a polymer matrix. In other embodiments, the composition is absorbable. In certain embodiments, the composition comprises a pH buffering agent. In some embodiments, the composition contains a lubricity enhancing agent.

In certain embodiments, the solubility of the chemically modified heparin may need to be enhanced. In such cases, the solubility may be increased by the use of appropriate formulation techniques, such as the incorporation of solubility-enhancing compositions such as mannitol, ethanol, glycerin, polyethylene glycols, propylene glycol, poloxomers, and others known in the art.

In certain embodiments, the composition contains a lubricity enhancing agent. As used herein, lubricity enhancing agents refer to one or more pharmaceutically acceptable polymeric materials capable of modifying the viscosity of the pharmaceutically acceptable carrier. Suitable polymeric materials include, but are not limited to: ionic and non-ionic water soluble polymers; hyaluronic acid and its salts, chondroitin sulfate and its salts, dextrans, gelatin, chitosans, gellans, bioconjugates or polysaccharides, or any combination thereof; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; collagen and modified collagens; galactomannans, such as guar gum, locust bean gum and tara gum, as well as polysaccharides derived from the foregoing natural gums and similar natural or synthetic gums containing mannose and/or galactose moieties as the main structural components (e.g., hydroxypropyl guar); gums such as tragacanth and xanthan gum; gellan gums; alginate and sodium alginate; chitosans; vinyl polymers; hydrophilic polymers such as polyethylene oxides, polyoxyethylene-polyoxypropylene copolymers, and polyvinylalcohol; carboxyvinyl polymers or crosslinked acrylic acid polymers such as the “carbomer” family of polymers, e.g., carboxypolyalkylenes that may be obtained commercially under the Carbopol™ trademark; and various other viscous or viscoelastomeric substances.

Suitable pH buffering agents for use in the compositions herein include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or MES. In certain embodiments, an appropriate buffer system (e.g., sodium phosphate, sodium acetate, sodium citrate, sodium borate or boric acid) is added to the composition to prevent pH drift under storage conditions. In some embodiments, the buffer is a phosphate buffered saline (PBS) solution (i.e., containing sodium phosphate, sodium chloride and in some formulations, potassium chloride and potassium phosphate). The particular concentration will vary, depending on the agent employed. In certain embodiments, the pH buffer system (e.g., sodium phosphate, sodium acetate, sodium citrate, sodium borate or boric acid) is added to maintain a pH within the range of from about pH 4 to about pH 8, or about pH 5 to about pH 8, or about pH 6 to about pH 8, or about pH 7 to about pH 8. In some embodiments, the buffer is chosen to maintain a pH within the range of from about pH 4 to about pH 8. In some embodiments, the pH is from about pH 5 to about pH 8. In some embodiments, the buffer is a saline buffer. In certain embodiments, the pH is from about pH 4 and about pH 8, or from about pH 3 to about pH 8, or from about pH 4 to about pH 7.

The chemically modified heparin or composition comprising the same may be sterilized to remove unwanted contaminants including, but not limited to, endotoxins and infectious agents. Sterilization techniques which do not adversely affect the structure and biotropic properties of the chemically modified heparin can be used. In certain embodiments, the chemically modified heparin can be disinfected and/or sterilized using conventional sterilization techniques including propylene oxide or ethylene oxide treatment, sterile filtration, gas plasma sterilization, gamma radiation, electron beam, and/or sterilization with a peracid, such as peracetic acid. In one embodiment, the chemically modified heparin can be subjected to one or more sterilization processes. Alternatively, the chemically modified heparin may be wrapped in any type of container including a plastic wrap or a foil wrap, and may be further sterilized.

In some embodiments, separate or sequential administration of the chemically modified heparin or composition comprising the same is necessary to facilitate delivery. In certain embodiments, the chemically modified heparin or composition comprising the same can be administered at different dosing frequencies or intervals. Additionally, as will be apparent to those skilled in the art, the chemically modified heparin or composition comprising the same can be administered using the same route of administration or different routes of administration.

In one embodiment, the treatment methods can further include administration of an effective amount of another agent. In some embodiments, the other agent is an anti-spike protein antibody or fragment. In some embodiments, the second agent is co-administered with the antibody or fragment thereof simultaneously or sequentially.

In some embodiments, the second agent is effective in reducing or inhibiting cytokine release storm. In some embodiments, the second agent is a corticosteroid. Non-limiting examples include methylprednisolone (in particular in patients with a rheumatic disease) and dexamethasone (in particular in patients with FHLH).

In some embodiments, the second agent is a cytoablative therapy. Non-limiting examples include cyclophosphamide (in particular in patients with JIA and MAS), etoposide (in particular in patients with FHLH), rituximab (in particular in Epstein-Barr virus (EBV)-associated HLH), antithymocyte globulin (in particular for patients at bone marrow transplant phase of FHLH therapy), and alemtuzumab (in particular in patients with FHLH or SLE-associated MAS).

In some embodiments, the second agent is a T-cell modulator. Non-limiting examples include calcineurin (e.g., cyclosporine), which prevents production of IL-2, and abatacept, which inhibits CD28 signaling of T cells. In some embodiments, the second agent is an anti-GM-CSF inhibitor or antibody.

In some embodiments, the second agent is a cytokine inhibitor, such inhibitors targeting INFγ, IL-1β, IL-18, IL-33, IL-6, and/or TNF.

In some embodiments, the second agent is a chemotherapeutic agent. Exemplary chemotherapeutic agents include, but are not limited to, cisplatin, etoposide, irinotecan, camptostar, topotecan, paclitaxel, docetaxel, epothilones, taxotere, tamoxifen, 5-fluorouracil, methoxtrexate, temozolomide, cyclophosphamide, SCH 66336, R115777, L778,123, BMS 214662, IRESSA® (gefitinib), TARCEVAR® (erlotinib hydrochloride), antibodies to EGFR, GLEEVEC® (imatinib), intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, pentostatine, vinblastine, vincristine, vindesine, bleomycin, doxorubicin, dactinomycin, daunorubicin, epirubicin, idarubicin, mithramycin, deoxycoformycin, Mitomycin-C, L-Asparaginase, teniposide, 17α-Ethinylestradiol, Diethylstilbestrol, testosterone, prednisone, fluoxymesterone, dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesterone acetate, leuprolide, flutamide, toremifene, goserclin, carboplatin, hydroxyurca, amsacrine, procarbazinc, mitotane, mitoxantronc, levamisole, navelbene, anastrazole, letrazole, capecitabine, reloxafine, droloxafine, hexamethylmelamine, Avastin, herceptin, Bexxar, Velcade, Zevalin, Trisenox, Xeloda, Vinorelbine, Porfimer, Erbitux® (cetuximab), Liposomal, Thiotepa, Altretamine, Melphalan, Trastuzumab, Lerozole, Fulvestrant, Exemestane, Fulvestrant, Ifosfomide, Rituximab, C225, Campath, carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, paclitaxel, gemcitabine, navelbine, farnesyl-protein transferase inhibitors, transplatinum, 5-fluorouracil, vincristine, vinblastine, and methotrexate.

In certain embodiments, the second agent is a CDK (cyclin-dependent kinase) inhibitor, such as ribociclib, palbociclib, abemaciclib, P1446A-05, trilaciclib, favopiridol, olomucine, roscovitine, dinaciclib, PD-0332991, SNS-032, LY-2835219, R547, LEE011, AT7519, AZD5438, or AG-024322.

In certain embodiments, the second agent is a checkpoint inhibitor. Exemplary checkpoint inhibitors include, but are not limited to, ipilimumab (Yervoy®), nivolumab (Opdivo®), pembrolizumab (Keytruda®), atczolizumab (Tecentriq®), avelumab (Bavencio®), durvalumab (Imfinzi®), and cemiplimab (Libtayo®).

In certain embodiments, the second agent is a chimeric antigen receptor T cell (CAR-T cell). Exemplary CAR-T cells include, but are not limited to, tisagenlecleucel (Kymriah®), axicabtagene ciloleucel (Yescarta®), brexucabtagene autoleucel (Tecartus®), lisocabtagene maraleucel (Breyanzi®), and idecabtagene vicleucel (Abecma®).

In some embodiments, the second agent is a viral vector, such as those which are used for gene therapy. Exemplary viral vectors include, but are not limited to, those associated with retroviruses, lentiviruses, adenoviruses, adeno-associated viruses (AAVs), plant viruses, or a hybrids thereof. In some embodiments, the second agent is a bacteriophage (e.g. Qβ, AP205).

As such, in certain embodiments, provided herein is a method for the treatment of cancer, which includes administering to a subject in need of treatment a therapeutically-effective amount of a chemically modified heparin or composition comprising the same as described herein in combination with one or more chemotherapeutic agents.

Administration of the chemically modified heparin or composition comprising the same as described herein may precede or follow the second agent or treatment by intervals ranging from minutes to weeks. For example, in certain aspects, one or more agents may be administered within about 1 minute, about 5 minutes, about 10 minutes, about 20 minutes about 30 minutes, about 45 minutes, about 60 minutes, about 2 hours, about 3 hours, about 4 hours, about 6 hours, about 8 hours, about 9 hours, about 12 hours, about 15 hours, about 18 hours, about 21 hours, about 24 hours, about 28 hours, about 31 hours, about 35 hours, about 38 hours, about 42 hours, about 45 hours, to about 48 hours or more prior to and/or after administering the chemically modified heparin or composition comprising the same. In certain embodiments, an agent may be administered within from about 1 day, about 2 days, about 3 days, about 4 days, about 5 days, about 6 days, about 8 days, about 9 days, about 12 days, about 15 days, about 16 days, about 18 days, about 20 days, to about 21 days prior to and/or after administering the chemically modified heparin or composition comprising the same. In some situations, it may be desirable to extend the time period for treatment significantly, however, where several weeks (e.g., about 1, about 2, about 3, about 4, about 6, or about 8 weeks or more) lapse between the respective administrations.

In some embodiments, the second agent targets the underlying disease or condition, such as SARS-CoV-2 infection. Non-limiting examples include lopinavir, ritonavir, oseltamivir (Tamiflu), favipiravir, fingolimod, methylprednisolone, bevacizumab, chloroquine phosphate, chloroquine, hydroxychloroquine sulfate, and remdesivir.

In another aspect, the present disclosure provides a pharmaceutical composition comprising a heparin of the present disclosure formulated together with a pharmaceutically acceptable carrier. It may optionally contain one or more additional pharmaceutically active ingredients, such as a heparin or a drug. The pharmaceutical compositions of the disclosure also can be administered in a combination therapy with, for example, an anti-viral agent, or a vaccine.

The pharmaceutical composition can comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference. In certain embodiments, a pharmaceutical composition is suitable for intravenous, intramuscular, subcutaneous, parenteral, spinal, intravitreal, or epidermal administration (e.g., by injection or infusion). Depending on the route of administration, the active compound can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intravitreal, and intrasternal injection and infusion. Alternatively, a heparin of the disclosure can be administered via a non-parenteral route, such as a topical, epidermal or mucosal route of administration, e.g., intranasally, orally, vaginally, rectally, sublingually or topically.

For administration by inhalation or intranasal routes, the chemically modified heparin can be delivered in the form of a solution, suspension, emulsion, or semisolid aerosol from pressurized packs, or a nebuliser, usually with the use of a propellant, e.g., halogenated carbons derived from methane and Ethan, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Pharmaceutical compositions can be in the form of sterile aqueous solutions or dispersions. They can also be formulated in a microemulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient, or from about 0.1% to about 70%, or from about 1% to about 30% of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used chemically modified heparin refers to physically discrete units suited as unitary dosages for the subjects to be treated; each unit contains a predetermined quantity of active compound calculated to produce the desired therapeutic effect in association with the required pharmaceutical carrier. Alternatively, the chemically modified heparin can be administered as a sustained release formulation, in which case less frequent administration is required.

For administration of the chemically modified heparin, the dosage ranges from about 0.0001 to 100 mg/kg, or about 1 to 12 mg/kg, or about 1 to 6 mg/kg, or 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per day, twice per day, once per week, once every two weeks, once every three weeks, once every four weeks, once a month, once every 3 months or once every 3 to 6 months. An exemplary dosage regimen for a chemically modified heparin of the disclosure includes 1 mg/kg body weight, 3 mg/kg body weight, or up to 6 mg/kg body weight via intravenous or subcutaneous administration. An exemplary dosage regimen for a chemically modified heparin of the disclosure includes 1 mg/kg body weight, 3 mg/kg body weight, or up to 6 mg/kg body weight via intravenous administration, with the chemically modified heparin being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma heparin concentration of about 1-1000 μg/mL and in some methods about 25-300 μg/mL.

An exemplary dosage regimen for a subject in vaso-occlusive crisis for a chemically modified heparin of the disclosure includes 3 mg/kg (say 1-6 mg/kg) twice daily by s.c. route and for a duration of 3-10 days during the vaso-occlusive crisis.

In some methods, dosage is contemplated as twice daily dosing via subcutaneous delivery. In some methods, dosage is contemplated as twice daily dosing via subcutaneous delivery at 10 mg/kg, for about 3-5 days, or up to 10 days (e.g., for sickle-cell disease), or chronically for up to several months (e.g., for cancer).

In some embodiments, a suitable dose of a chemically modified heparin of the disclosure for a human patient is from 5 mg to 1200 mg, from 10 mg to 1000 mg, from 20 mg to 500 mg, from 20 mg to 300 mg, from 20 mg to 200 mg, from 50 mg to 150 mg, from 70 mg to 120 mg daily. In some embodiments, a suitable dose of a heparin of the disclosure for a human patient is about 5 mg, 10 mg, 20 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg daily. In some embodiments, a suitable dose of a chemically modified heparin of the disclosure for a subject is about 100 mg daily.

In some embodiments, the chemically modified heparin is administered to a subject from 0.2 mg/Kg/hour to 50 mg/Kg/hour, from 0.4 mg/Kg/hour to 40 mg/Kg/hour, from 0.8 mg/Kg/hour to 30 mg/Kg/hour, from 2 mg/Kg/hour to 30 mg/Kg/hour, from 4 mg/Kg/hour to 30 mg/Kg/hour, from 6 mg/Kg/hour to 30 mg/Kg/hour, from 8 mg to 25 mg/Kg/hour, from 12 mg/Kg/hour to 20 mg/Kg/hour, from 0.2. mg to 25 mg/Kg/hour, from 0.2 mg/Kg/hour to 20 mg/Kg/hour, from 0.2 mg/Kg/hour to 15 mg/Kg/hour, from 0.2 mg/Kg/hour to 12 mg/Kg/hour, from 0.2 mg/Kg/hour to 8 mg/Kg/hour, from 0.2 mg/Kg/hour to 5 mg/Kg/hour, or from 0.2 mg/Kg/hour to 2 U/Kg/hour by continuous infusion. In some embodiments, a suitable dose of a heparin of the disclosure for a human patient is about 0.1 mg/Kg/hour, 0.2 mg/Kg/hour, 0.3 mg/Kg/hour, 0.4 mg/Kg/hour, 0.5 mg/Kg/hour, 0.6 mg/Kg/hour, 0.7 mg/Kg/hour, 0.8 mg/Kg/hour, 0.9 mg/Kg/hour, 1 mg/Kg/hour, 2 mg/Kg/hour, 3 mg/Kg/hour, 4 mg/Kg/hour, 5 mg/Kg/hour, 6 mg/Kg/hour, 7 mg/Kg/hour, 8 mg/Kg/hour, 9 mg/Kg/hour, 10 mg/Kg/hour, 15 mg/Kg/hour, or 20 mg/Kg/hour, by continuous infusion. In some embodiments, the chemically modified heparin is administered to a subject from 0.2 U/Kg/hour to 2 U/Kg/hour by continuous infusion.

In some embodiments, the dosage regimen includes a loading dose, followed by a maintenance dose (mg/kg/h) to achieve the appropriate dosage range (e.g., 1 to 12 mg/kg/day). In some embodiments, the loading dose is administered intravenously as a bolus. In some embodiments, the loading dose is administered intravenously as an infusion.

In some embodiments, the dosage regimen comprises an intravenous loading dose of from about 0.1 to 100 mg/kg, following by a continuous dose of from about 0.01 to 10 mg/kg/h.

In some embodiments, the administration is once, twice, or three times a day. In some embodiments, the administration is once a week or once a month.

### EXAMPLES

### Example 1. Chemically Modified Heparin

Utilizing carbodiimide chemistry, the carboxyl groups on the iduronic/glucuronic saccharides of heparin were chemically modified as described below. The reaction of carbodiimide with a carboxyl group generally proceeds through the addition of the free carboxylate to one of the double bonds of the diimide to give an O-acylurea product. In the absence of a nucleophile the O-acylurea to rearranges to the more stable N-acylurea through an intramolecular acyl-transfer.

Bovine intestinal heparin was reacted with N-(3-dimethylaminopropyl)-N′-ethylcarbodiimide hydrochloride (EDAC) at varying molar ratios of EDAC to the carboxylate groups on heparin (—COOH). Information on heparin, EDAC, and reaction buffer are described in Table 1.

TABLE 1
EDAC and heparin reaction materials

| Parameter | Value | Units |
|---|---|---|
| Heparin^{†} MW | 16000 | Da |
| # —COOH/heparin^{†} (600 Da/disaccharide) | 27 | —COOH/Heparin |
| MW EDAC | 191.7 | Da |
| Concentration of heparin stock solution | 20 | mg/mL |
| pH | 4.5 | |
| Buffer | MES | |

^{†}Unfractionated non-chemically modified bovine intestinal heparin

The heparin was dissolved into MES buffer at 20 mg/mL. EDAC dry powder was added directly to the heparin solution and was dissolved by vortexting at the molar ratios listed in Table 2. The reaction was then carried out for 1 hour at room temperature (74° F.). The modified heparin was then purified by tangential flow filtration using a 10 kDa MWCO mPES filter (Repligen). At least 3 volumes of 300 mM NaCl, followed by at least 10 volumes of water were exchanged in the purification process to yield purified chemically modified heparin compounds in water.

Concentration of test compound was determined by SEC-HPLC method with running conditions as described by the heparin USP. The anticoagulant activity of the compounds was measured by anti-factor IIa activity per USP methods. Briefly, anti-factor IIa activity was determined as follows. Each sample/standard was run in duplicate. 50 μL standard/sample/blank (reaction buffer, 0.05M tris) was pipetted into each well. 100 μL Working Solution Reagent R1 (antithrombin) was added to each well, and incubated at 37° C. for 2 minutes at 900 rpm. 25 μL Working Solution Reagent R2 (thrombin) was added to each well and incubated at 37° C. for 2 minutes at 900 rpm. 50 μL Working Solution Reagent R3-(chromogenic substrate) was added to each well and incubated at 37° C. for 1 minute at 900 rpm. 50 μL Stopping Solution was added to each well and incubated at 37° C. for 1 minute at 900 rpm. The absorbance of the samples/standards was measured using the plate reader (405 nm) against the blank.

Sample dilution was adjusted due to the lower potency of the test Compound samples. Heparin concentration was determined previously using HPLC-RI.

Samples were analyzed using microtiter plate method using endpoint measurement based on USP <208>.

As shown in Table 2, the anticoagulant activity of the chemically modified heparin was reduced as compared to the parent non-chemically modified bovine intestinal heparin, where complete removal of anticoagulation was achieved at a molar ratio of 2:1 (EDAC:hep-COOH).

TABLE 2

| | | | Bovine |
|---|---|---|---|
| | Compound A | Compound C | heparin^{†} |
| Mole EDAC per COOH group | 2.0 | 0.5 | 0.0 |
| Anti-Factor IIa activity (U/mg) | 0.2 | 28.9 | 107 |
| % of parent heparin Anti-factor | 0% | 27% | 100% |
| IIa | | | |

^{†}Unfractionated non-chemically modified bovine intestinal heparin

Functionalization was confirmed by HPLC-RI as no unconjugated EDAC, or unconjugated EDAC byproduct, was observed. HPLC Conditions: HPLC: Agilent 1100 HPLC with refractive index detector; Column: TSKgel G3000SW XL, 7.8×300 mm, 5 micron (Tosoh Bioscience, 08541)+TSKgel G4000SW XL, 7.8×300 mm, 8 micron (Tosoh Bioscience, 08542); Guard Column: TSKgel G2000SWxl-G4000SWxl, and QC-PAK GFC Guard Column for 7.8 mm ID columns, 7 micron (Tosoh Bioscience, 08543); Mobile Phase: 0.1M ammonium acetate in 0.02% azide; Detection: Refractive Index; Column Temperature: 30° C.; Flow Rate: 0.6 mL/min; Injection Volume: 20 μL.

| Sample | Retention Time (mean, n = 2) |
|---|---|
| Compound A | 30.08 |
| Compound C | 29.85 |

A calculation of the degree of functionalization (or degree of substitution) of modified N-acyl-urea modified heparins, Compound A and Compound C, was completed by comparing the anomeric proton region to a clear signal for the modifying side chain on the polymer backbone using NMR.

Nuclear magnetic resonance spectroscopy (NMR) was completed at 500 MHZ (30° C. and 70° C.) with D₂O as solvent. Spectra were referenced to the HDO peak (4.70 ppm for proton (30° C.)). For the 2-D spectra discussed here (without a carbon reference signal for the carbon domain) the acetate methyl resonance was found to resonate at ¹³C δ 22.0 ppm without adjusting the spectral reference.

Integration of the entire anomeric region for use as a comparison. The anomeric region was integrated using the region between about δ 4.94-6.0 ppm and assigned a value of 2-protons i.e. one disaccharide unit.

The integration of the resonance at δ 2.7-3.0 ppm was assigned as the two methyl resonances on the ammonium species (terminating the side chain) and so these 6 protons appear as a singlet. Integration for the anomeric region has been normalised to 2 protons representing the two protons on the anomeric centres of each disaccharide, therefore the degree of substitution (DOS), i.e. the number of dimethyl containing side-chains per disaccharide, may be calculated by dividing the methyl resonance by 6.

The degree of substitution for Compound A and Compound C is shown in Table 3.

TABLE 3

| | Degree of Substitution |
|---|---|
| Compound | (DOS) |
| Compound A | 0.57 |
| Compound C | 0.13 |

The data indicates that for Compound C, approximately 1 in 8 of the carboxyl's has reacted, while for Compound A, just over half of the carboxyl's have been substituted. The assignment of the key resonances of the acyl urea side chain was confirmed by 2-D NMR spectroscopy. HSQC experimental data confirmed that the anomeric region does not appear to be confounded as the degree of substitution changes.

### Example 2. Mouse Model with LPS Activation of Inflammation

The following is an in vivo mouse model for evaluating test compounds for TNF-alpha and C5a inhibition.

Male Swiss Webster mice, 7-8 weeks of age, are acclimated for at least 7 days prior to study start. On study day −1, animals are weighed and randomized by body weight. In the evening of study day −1, animals are dosed with vehicle or test articles as described in Table 3. On study day 0, two minutes after the IV vehicle/test article dosing, animals are then dosed IP with saline (group 1) or LPS (1 mg/kg). At 2 hours post LPS dose (at corresponding 1 minute intervals), animals are sacrificed by inhaled isoflurane anesthesia, exsanguination and then a cervical dislocation to confirm euthanasia. Serum are collected in 3×60 μL aliquots for each animal and stored at −80° C. until further testing. TNF-alpha and C5a concentrations from serum samples are measured by ELISA.

### Example 3. P-Selectin-Mediated Cell Binding

The following example shows that the compounds disclosed herein inhibit P-selectin, demonstrated by inhibition of neutrophil-like cell binding.

### In Vitro Study Design

96 well plates were coated with 10 μg/mL Protein A overnight, then blocked with 2% FBS for 1 hour. 2 μg/mL P-selectin/Fc chimera was bound to the Protein A for 3 hours at 4° C. HL-60 cells (2e5 cells/well, CMFDA labeled) were then layered onto the P-selectin and allowed to bind for 1 hour at room temperature. Wells were treated concurrently with test samples to determine inhibitory binding activity. After 1 hour, unbound cells were washed and bound cells lysed in a 1% Triton-X solution and read on a Fluorimeter at 480/520 nm.

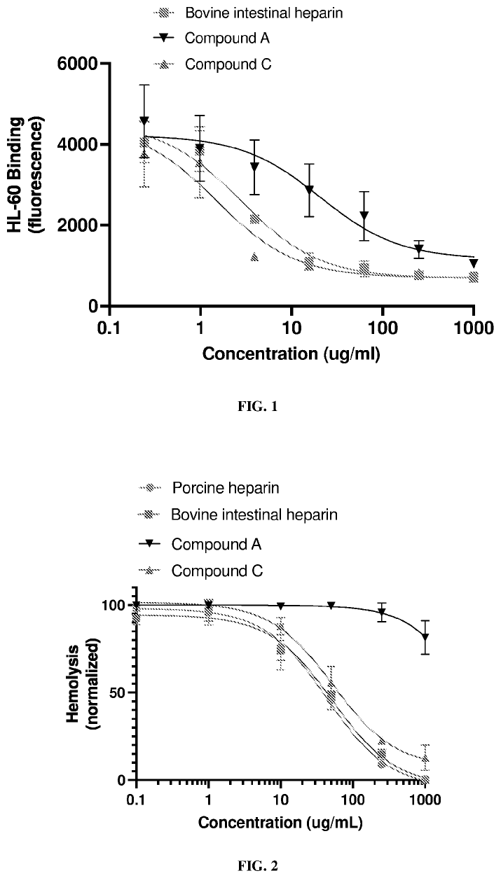

As shown in FIG. 1, compounds as provided herein inhibit P-selectin-mediated neutrophil (HL-60) cell binding. P-selectin IC₅₀ values for unfractionated non-chemically modified porcine heparin, non-chemically modified bovine intestinal heparin, enoxaparin, Compound A, and Compound C are shown below in Table 4.

TABLE 4

| Best-fit | Porcine | Bovine | Enoxa- | Compound | Compound |
|---|---|---|---|---|---|
| values | Heparin | Heparin^{†} | parin | A | C |
| P-sel IC₅₀ | 2.013 | 1.98 | 64.72 | 15.1 | 1.5 |
| (μg/mL) | | | | | |
| Anti-IIa | 217 | 107 | N/A | 0.2 | 29 |
| (U/mL) | | | | | |
| Complement | 43 | 52 | Not | No | 53 |
| IC₅₀ (μg/mL) | | | tested | activity | |

^{†}Unfractionated non-chemically modified bovine intestinal heparin

### Example 4. In Vitro Complement Inhibition Studies

Complement, an important effector mechanism of the immune system, is an enzymatic cascade of approximately 30 serum proteins leading to the amplification of a specific humoral response. It can be activated through the classical or alternative pathways, or through the mannose-binding lectin pathway. Deficient or exacerbated activation of the complement system leads to diseases of variable severity, and pharmacological inhibition of the complement system is considered as a therapeutic strategy to ameliorate the inflammatory effects of exacerbated complement activation.

Compounds disclosed herein were tested and for complement inhibition using the total complement assay, CH50, as described in Oberkersch et al. Thrombosis research 125.5 (2010): e240-e245. Briefly, pooled normal serum from healthy subjects was incubated with increasing concentrations of test compounds in the presence of sensitized sheep erythrocytes (EA). The total complement activity in samples was determined spectrophotometrically by measuring the extent of hemolysis. Test compounds were evaluated at five different concentrations in triplicate to determine the extent of hemolysis. The experiments were performed in three (3) independent runs for evaluating the effects of the test compounds. The IC₅₀ values corresponding to the test articles for the inhibition of the classical complement pathway were calculated from the dose response curves plotted using the test article concentration vs the % of complement induced hemolysis. IC₅₀s for complement inhibition of non-chemically modified porcine heparin, non-chemically modified bovine intestinal heparin, Compound A, and Compound C are shown in Table 5.

TABLE 5

| Complement inhibition (CH50) | IC₅₀ (μg/mL) |
|---|---|
| Porcine Heparin | 43 |
| Bovine Heparin^{†} | 52 |
| Compound A | No activity |
| Compound C | 53 |

^{†}Unfractionated non-chemically modified bovine intestinal heparin

FIG. 2 shows hemolysis data for non-chemically modified porcine heparin, non-chemically modified bovine intestinal heparin, Compound A, and Compound C. As shown in this example, complement inhibition as measured in the CH50 assay above for chemically modified bovine intestinal heparin was diminished.

### Example 5: In Vivo Inhibition of Melanoma Metastasis

In vivo inhibition of melanoma metastasis is evaluated as follows. Six week old female C57/BL6 mice (Charles River) are injected with luciferase-expressing mouse melanoma cells (B16/F10-luc) at 3×10⁵ cells in 100 μL through intravenous tail vein injection. Mice are pretreated with 100 μL intraperitoneal injection of either saline (control) or test compound 30 minutes prior to administration of melanoma cells. Metastasis and tumor formation in the lungs of mice is measured at various time points by in vivo imaging using bioluminescence of luciferase reporting cells by administration of 10 mg/kg D-luciferin. Fluorescence is quantified by measuring the region of interest in the lungs. Data are presented from 7-days post-administration of melanoma cells, where inhibition of fluorescence indicates inhibition of lung metastasis.

### Example 6: HL-60 Cell Assay

HUVECs are cultured on custom flow channels coated with fibronectin. Following confluent layer (24-48 hour) formation, HUVECs are treated with TNF-alpha (10 ng/ml) for 4 hours. HL-60 cells are then injected and perfused for 10-15 minutes to allow for adhesion to the activated endothelium. Test compound is added to HUVECs at 100 μg/mL during incubation period with TNF-alpha while cells with medium alone served as the negative control. Bound HL-60 cells are then quantified by microscopy imaging and compared to control channels. Experiments are performed in independent triplicate. Inhibition of cell binding to inflamed endothelium supports potential to inhibit selectin-mediated cell binding to selectin-expressing cells, such as the endothelium or an inflamed endothelium. This mechanism supports the potential for test compound to prevent sickle cell adhesion, and thus supports use of the compounds disclosed herein for treating sickle cell disease, and as a rescue medication for sickle cell disease patients in the prodromal phase of vaso-occlusive crisis.

### Example 7: Rat PD Study

Compounds are evaluated for pharmacodynamics when administered subcutaneously (SC) or intravenously (IV) in male and female Sprague Dawley rats as follows. On study day −1, the animals are randomized by body weight into treatment groups. On study day 0, the animals are administered a single (1×) IV or SC dose of test compound at 30 mg/kg (15 mg/mL). The animals are bled via the tail vein for serum at 8 time points (pre-dose through 8 h post-dose) then bled terminally via descending aorta blood draw and euthanized at 8 hours post-dose (Group 1, dosed IV) or 24 hours post-dose (Group 2, dosed SC). Serum samples are analyzed for HL-60 binding to P-selectin using a custom HL-60/P-selectin Binding Assay (as described herein) with the readout being fluorescence intensity from the labeled cells.

### Example 8: Pancreatic Cancer BxPC3 Model

Compounds are evaluated for anti-cancer activity in a mouse model of pancreatic cancer as follows. Human pancreatic cancer cells, BxPC3, are implanted subcutaneously (1×10⁶ cells in 100 μL Matrigel) in 6 week old female nude mice (5 mice per group). Treatment begins on day 7 after implantation, with control (e.g., gemcitabine at 100 mg/kg i.p. q3 day) or test compound (5 mg/kg s.c. bid). Tumor volume is measured with calipers each week. On day 56 the dose of test compound is increased to 10 mg/kg s.c. bid. At day 70 animals are sacrificed and tumors resected, fixed, and stored for further testing.

### Example 9: Gemcitabine Sparing Pancreatic Cancer Model

Methods: Pancreatic cancer tumors were generated by subcutaneous injection of 100 μL of BxPC3-luc, clone 1F11 cells (1×10⁶) in matrigel into the right flank of female NSG mice. Tumors were allowed to grow for 7 days prior to initiating treatment. At day 7 post-implant, mice were randomized into treatment groups (n=5/group) and treatment was initiated per dose route and regimen as described below:

| Group | Treatment | Route of Administration |
|---|---|---|
| 1 | Control (PBS) | subcutaneous |
| 2 | Compound C | subcutaneous |
| 3 | Compound C + Gemcitabine | subcutaneous + intraperitoneal |
| 4 | Gemcitabine | subcutaneous |

Gemcitabine (Gem) at a low sensitizing dose was administered 25 mg/kg QW by intraperitoneal route. Compound C was administered 10 mg/kg BID by subcutaneous route. Treatments were administered from day 7 through day 70.

Body weight and tumor volume by caliper was measured weekly. At day 70 imaging was performed by administering 150 mg/kg luciferin and measuring whole body fluorescence by IVIS imaging system. Tumors and organs were then harvested and imaged ex vivo.

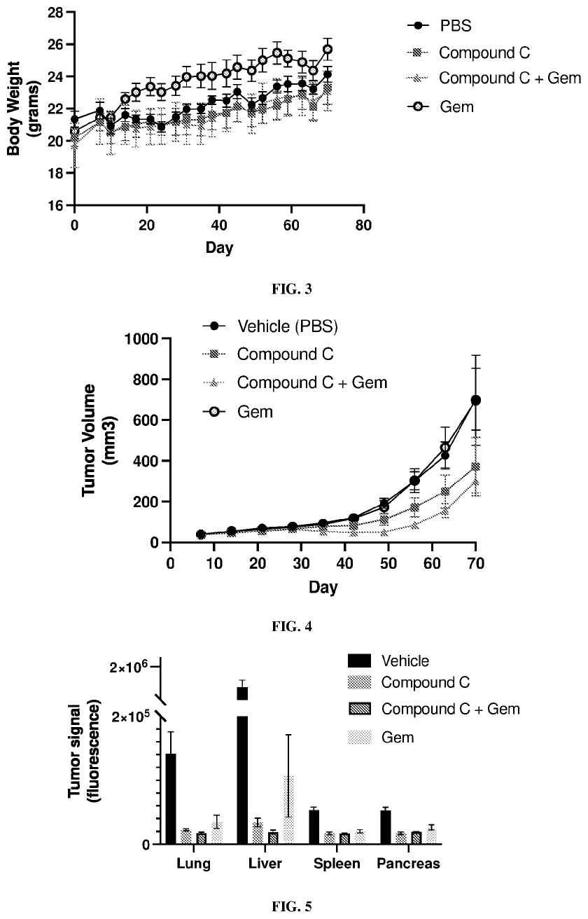

Results: All animals survived through the 70 day study period and treatment was well-tolerated in all groups. Body weight increased in all groups, as shown in FIG. 3.

As shown in FIG. 4, tumor volume increased among all groups and tumor growth was inhibited by about 50% by day 70 in treatment groups with the most pronounced tumor growth inhibition observed with combination treatment of gemcitabine+Compound C with low sensitizing dose of gemcitabine. Single agent treatment with Compound C also inhibited tumor growth. Gemcitabine alone had no effect on tumor growth.

Metastasis to other organs was measured by ex vivo fluorescence. As shown in FIG. 5 the organs with highest metastasis were the lungs and liver. Inhibition of metastasis was observed with all treatment groups, with trends for increased metastasis inhibition with Compound C treatment.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A chemically modified bovine intestinal heparin comprising from about 15 to about 90 disaccharide units, wherein about 2.5% to 14% of the disaccharide units comprise a 1-(3-dimethylaminopropyl)-3-ethylurea (EDU)-amide; and
the anti-factor IIA activity is about 20 to about 30 IU/mg.

2. The chemically modified bovine intestinal heparin of claim 1, wherein the P-selectin activity is not substantially different than the parent non-chemically modified bovine intestinal heparin.

3. The chemically modified bovine intestinal heparin of claim 1, wherein the P-selectin IC₅₀ is less than about 5 μg/mL.

4. A chemically modified bovine intestinal heparin of Formula IA: 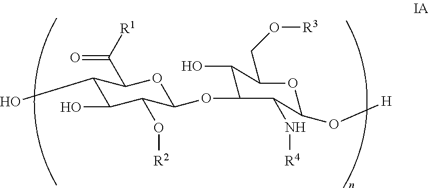
or a salt thereof, wherein:
n is 26-30;
each R¹ is independently —OH, 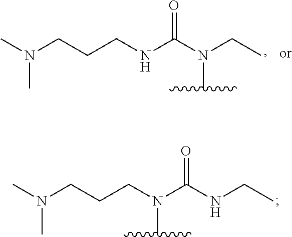
each R² is independently hydrogen, —S(O)₂O⁻, —S(O)₂OH, or —S(O)₂OM;
each R³ is independently hydrogen, —S(O)₂O⁻, —S(O)₂OH, or —S(O)₂OM;
each R⁴ is independently hydrogen, —S(O)₂O⁻, —S(O)₂OH, or —S(O)₂OM; and
each M is independently a cation; wherein about 5 to about 13.5% of the R¹ moieties are either

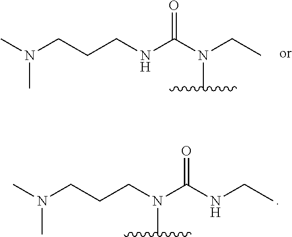

5. A composition comprising the chemically modified bovine intestinal heparin of any preceding claim.

6. A pharmaceutical composition comprising a chemically modified bovine intestinal heparin, wherein at least a portion of free carboxylic acid moieties on a non-chemically modified bovine intestinal heparin have been converted to a N-acylurea amide such that the chemically modified bovine intestinal heparin exhibits between 20% and 50% of the anti-factor IIa activity of the non-chemically modified bovine intestinal heparin; and
wherein the complement inhibition activity of the chemically modified bovine intestinal heparin is substantially the same as that of the non-chemically modified bovine intestinal heparin.

7. The pharmaceutical composition of claim 6, wherein the chemically modified bovine intestinal heparin comprises one or more chemically modified saccharide units of Formula I: 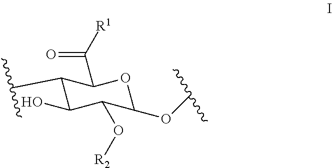
wherein:
each R¹ is independently 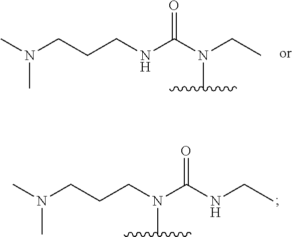
and
R² is hydrogen, —S(O)₂O⁻, —S(O)₂OH, or —S(O)₂OM; where M is a cation.

8. The pharmaceutical composition of claim 7, wherein R² is hydrogen, —S(O)₂O⁻, —S(O)₂OH, or —S(O)₂ONa.

9. The pharmaceutical composition of claim 7, wherein the chemically modified bovine intestinal heparin comprises one or more chemically modified saccharide units of Formula IIA: 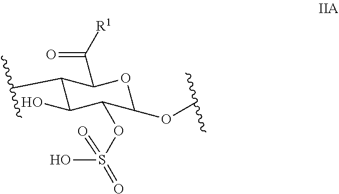
wherein each R¹ is independently

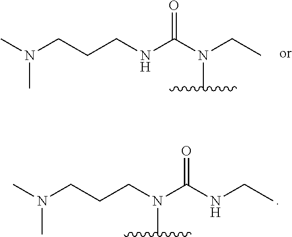

10. The pharmaceutical composition of claim 7, wherein the chemically modified bovine intestinal heparin comprises one or more chemically modified saccharide units of Formula IIB: 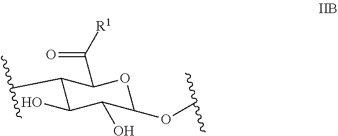
wherein each R¹ is independently

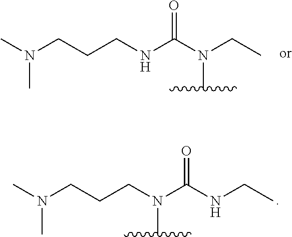

11. The pharmaceutical composition of any one of claims 6-10, wherein the chemically modified bovine intestinal heparin has an anti-factor IIa activity between 80 U/mg and 135 U/mg.

12. The pharmaceutical composition of any one of claims 6-11, wherein the pharmaceutical composition exhibits from about 25% to about 35%, of the anti-factor IIa activity of the non-chemically modified bovine intestinal heparin.

13. The pharmaceutical composition of any one of claims 6-12, wherein the chemically modified bovine intestinal heparin is unfractionated bovine intestinal heparin.

14. The pharmaceutical composition of any one of claims 6-13, wherein the P-selectin inhibitory activity is not significantly diminished as compared to the non-chemically modified bovine intestinal heparin.

15. The pharmaceutical composition of any one of claims 6-13, wherein the P-selectin inhibitory activity (IC₅₀) is between less than 150% and greater than 150% that of the non-chemically modified bovine intestinal heparin.

16. The pharmaceutical composition of any one of claims 6-13, wherein the P-selectin inhibitory activity is substantially the same as that of the non-chemically modified bovine intestinal heparin.

17. A method for reducing inflammation in a subject in need thereof, comprising administering to the subject an effective amount of the composition of claim 5, or pharmaceutical composition of any one of claims 6-16.

18. A method for treating or lessening one or more symptoms of sickle cell disease in a subject in need thereof, comprising administering to the subject an effective amount of the composition of claim 5, or pharmaceutical composition of any one of claims 6-16.

19. The method of claim 18, wherein the subject in need thereof is in the prodromal, or early phase of vaso-occlusive crisis.

20. A method for preventing or reversing cellular adhesion in a subject in need thereof, comprising administering to the subject an effective amount of the composition of claim 5, or pharmaceutical composition of any one of claims 6-16.

21. A method for preventing or reversing complement activation in a subject in need thereof, comprising administering to the subject an effective amount of the composition of claim 5, or pharmaceutical composition of any one of claims 6-16.

22. A method for treating a solid tumor in a subject in need thereof, comprising administering to the subject an effective amount of the composition of claim 5, or pharmaceutical composition of any one of claims 6-16.

23. The method of claim 22, wherein the solid tumor expresses at least one of Sialyl Lewis^{x} or Sialyl Lewis^{a} (sLex or sLea).

24. The method of claim 22, wherein the solid tumor is a gastrointestinal, breast, prostate, ovarian, colorectal, liver, lung, cervical, head, neck, esophageal, brain, or pancreatic tumor.

25. A method for treating a disease or disorder mediated at least in part by inhibition of cell binding to P-selectin and/or inhibition of a complement activation pathway in a subject in need thereof, comprising administering to the subject an effective amount of the composition of claim 5, or pharmaceutical composition of any one of claims 6-16.

26. The method of claim 25, wherein the disease or disorder is a cancer, a hematologic cancer, melanoma, leukemia, multiple myeloma, chemotherapy-induced peripheral neuropathy (CIPN), beta thalassemia, atypical hemolytic uremic syndrome (aHUS), paroxysmal nocturnal hemoglobinuria (PNH), a neurological disease, amyotrophic lateral sclerosis (ALS), sickle cell disease, vaso-occlusive crisis, immune response in gene therapy with adeno-associated virus (AAV), acute respiratory distress syndrome (ARDS), a cardiovascular disorder, an ophthalmological disease or disorder, a nephrological disorder, thrombogenic microangiopathy (TMA), hereditary angioedema, thrombotic thrombocytopenia purpura (TTP), Shiga toxin positive HUS, post-infection HUS, thrombotic microangiopathy, membranoproliferative glomerulonephritis (MPGN), primary MPGN, C3 glomerulopathy (C3G), transplant rejection, delayed kidney graft rejection, antibody-mediated kidney graft rejection, kidney graft reperfusion injury, kidney transplant in CAPS patients, neuromyelitis optica, multiple sclerosis, Guillain-Barré syndrome, myasthenia gravis, lupus nephritis, IgA nephropathy, rheumatoid arthritis, Crohn disease, ulcerative colitis, hemolytic anemia, autoimmune hemolytic anemia, pemphigus and pemphigoid, anti-phospholipid syndrome, cold agglutinin disease, severe thrombocytopenia, macular degeneration, uveitis, ANCA-associated vasculitis, atherosclerosis, mood disorders, asthma, chronic obstructive pulmonary disease (COPD), anaphylaxis, sepsis, cerebral malaria, psoriatic arthropathy, dermatomyositis, osteoarthritis, dementia, glaucoma, diabetic angiopathy, myocardial infarction, stroke, post-bypass, polytrauma, neurotrauma, antiphospholipid syndrome, preeclampsia, or hemodialysis.

27. The method of any one of claims 17-26, wherein the subject is on anticoagulant treatment.

28. The method of any one of claims 17-27, wherein the subject is human.

29. The method of any one of claims 17-28, wherein the administering comprises subcutaneous (SC) and intravenous (IV) administration.

30. The method of any one of claims 17-19, wherein the administering comprises intravenous (IV) administration.
